(19) United States
(12) Patent Application Publication
(10) Pub. No.: US 2008/0085243 A1
(43) Pub. Date: Apr. 10, 2008

(54) MOLECULAR MARKERS FOR DETERMINING TAXANE RESPONSIVENESS

(75) Inventors: Edward Weinstein, St. Louis, MO (US); Diana Ji, St. Louis, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: SIGMA-ALDRICH COMPANY, St. Louis, MO (US)

(21) Appl. No.: 11/867,812

(22) Filed: Oct. 5, 2007

Related U.S. Application Data
(60) Provisional application No. 60/828,269, filed on Oct. 5, 2006.

(51) Int. Cl. A61K 31/337; A61K 31/282; A61K 31/4162; A61K 31/437; A61K 31/675; A61K 31/7048; A61K 33/24; A61P 35/00; C12Q 1/68; G01N 33/15

(52) U.S. Cl. 424/9.2; 424/649; 435/6; 514/110; 514/27; 514/283; 514/411; 514/449; 514/492

(57) ABSTRACT

The present invention provides a plurality of molecular markers and methods of using the markers for determining whether a cancer will be responsive to a taxane. Also provided are kits comprising the plurality of molecular markers.

### CROSS-REFERENCE TO RELATED APPLICATION

This application claims priority to U.S. Provisional Application Ser. No. 60/828,269 filed on Oct. 5, 2006, which is hereby incorporated by reference in its entirety.

### FIELD OF THE INVENTION

The present invention relates to a plurality of molecular markers, methods, and kits for determining whether a cancer cell will respond to a taxane.

### BACKGROUND OF THE INVENTION

Cancer is a disease characterized by the uncontrolled growth and spread of abnormal cells. Around the world, over 10 million cancer cases occur annually. Half of all men and one-third of all women in the United States will develop some form of cancer during their lifetime. Cancer survival depends upon many factors, one of which is treatment with an effective chemotherapeutic agent or agents. One of the biggest challenges associated with cancer chemotherapy, however, is that patients with seemingly similar cancers do not respond the same way to a given agent. That is, some cancers respond to or are affected by an agent, whereas others are not affected by the agent.

Taxanes are a class of anticancer agents that are used to treat several different cancers, but with varying outcomes. For example, only about 20% of patients with late stage non-small cell lung cancer (NSCLC) respond therapeutically to the taxane, paclitaxel. Because of this low response rate, combinations of a taxane and another chemotherapeutic agent have been developed. But these multi-drug combinations have increased toxicity. What is needed, therefore, is a way of predicting the response of the cancer to a taxane before administering chemotherapy. Such a rational approach to chemotherapy would prevent patients from having to undergo chemotherapy treatments that will not have a clinically positive outcome. The method of predicting taxane responsiveness should be sensitive, easy to perform, and quick to provide an answer.

### SUMMARY OF THE INVENTION

Among the various aspects of the invention, therefore, is the provision of a method for determining whether a lung cancer will respond therapeutically to a taxane. The method comprises measuring the expression of at least one molecular marker in a cell from the cancer, and then assessing the responsiveness of the cancer to a taxane based upon the expression of the marker in the cancer cell relative to a control cell.

The invention also encompasses the set of molecular markers used for determining taxane responsiveness. The molecular markers comprise BRCA2, CDKN1C, CDKN2A, CYLD, DCC, DMBT1, FOS, GLTSCR2, HIC1, LATS1, LATS2, LZTS1, LZTS2, MSH2, NF1, PHB, PTEN, SMAD4, ST14, ST18, TGFBR2, TP53, TP73, TUSC2, TUSC5, VHL, and WT1.

Another aspect of the invention provides kits for determining whether a lung cancer will respond to a taxane. The kits comprise a plurality of agents for measuring the expression of at least one molecular marker in a cell from the cancer, wherein the expression of the molecular marker is altered in the cancer cell relative to a control cell.

Other aspects and features of the invention are described in more detail below.

### DESCRIPTION OF THE FIGURES

FIG. 1 illustrates the decreased expression of MDR1 in the MDR1-knockdown cells. Plotted is the percent of MDR1 expression as measured by quantitative real-time PCR for each MDR1 shRNA gene construct. All values were normalized to the empty pLKO.1 vector-transduced cells.

FIG. 2 illustrates the increased sensitivity of the MDR1 knockdown cells to paclitaxel. The mean absorbance at 450 nm, an indicator of cell survival, is plotted as a function of paclitaxel concentration for each of the different MDR1 constructs.

FIG. 3 depicts genes identified in the tumor suppressor screen whose down-regulation rendered tumor cells more responsive or less responsive to paclitaxel. The log of A₄₅₀ in the presence of 5 μM of paclitaxel is plotted versus the log of A₄₅₀ in the absence of drug for each shRNA-targeted gene. Lines were drawn through the points to help identify the outlying points. Points above the lines represent constructs that increased cell survival in the presence of paclitaxel, and points below the lines represent constructs that decreased cell survival in the presence of paclitaxel.

FIG. 4 illustrates genes whose knockdown conferred decreased cell survival in the presence of paclitaxel. Plotted is the relative cell survival of different shRNA constructs, as compared to the negative control pLKO.1, which was normalized to 1. Relative cell survival was calculated by determining the ratio of cell survival in the presence and absence of paclitaxel. The MDR1-2 construct served as a positive control.

FIG. 5 illustrates that multiple constructs to a single gene had similar effects on cell survival. Plotted is the relative cell survival of different shRNA constructs, as compared to the negative control pLKO.1, which was normalized to 1. Relative cell survival was calculated by determining the ratio of cell survival in the presence and absence of paclitaxel.

FIG. 6 illustrates genes whose knockdown conferred increased cell survival in the presence of paclitaxel. Plotted is the relative cell survival of different shRNA constructs, as compared to the negative control pLKO.1, which was normalized to 1. Relative cell survival is the ratio of cell survival in the presence and absence of paclitaxel.

### DETAILED DESCRIPTION OF THE INVENTION

The present invention provides a plurality of molecular markers that may be used to determine the responsiveness of a cancer cell to a class of chemotherapeutic agents, the taxanes. The molecular markers are genes whose altered expression in a cancer cell changes the response of the cancer cell to a taxane. The response of the cell may be either a therapeutic response (i.e., the drug will slow the rate of growth and/or lead to cell death) or a lack of a therapeutic response. Also provided herein are methods of using the molecular markers to determine whether a cancer cell will respond to a taxane, and kits for determining whether a cancer cell will respond to a taxane. The invention also provides compositions and methods for treating a cancer.

(I) Methods for Determining Whether a Cancer Cell Will Respond to a Taxane

One aspect of the invention provides a method for determining whether a cancer will respond to a taxane. The method comprises measuring the expression of at least one of the molecular markers of the invention in a cancer cell, wherein changes in the expression of the molecular marker in the cancer cell relative to a control cell indicate that the cancer will respond therapeutically to a taxane.

(a) Taxanes

Taxanes are a family of tricyclic diterpene alkaloids originally isolated from plants of the genus Taxus (yews). Taxanes have extremely complex structures; the basic core comprises a tricyclic taxane ring system in which twenty carbon atoms are arranged into three linked rings. Taxanes bind to the beta-subunit of tubulin and stabilize microtubules, thereby preventing microtubule depolymerization and arresting cells in mitosis. The tubulin-binding functional group of the taxane is contained within the tricyclic core of the molecule.

Taxanes comprise compounds having the core tricyclic taxane ring system. Taxanes may be natural, semi-synthetic, or synthetic. Taxanes comprise paclitaxel (TAXOL®), which was isolated from the bark of the Pacific yew tree, and docetaxel (TAXOTERE®) and baccatin III, which were derived from precursors extracted from the needles of yew trees. Taxanes also comprise precursors, derivatives, or analogs of paclitaxel, docetaxel, or baccatin III. Suitable taxanes include, but are not limited to, ortataxel (14 beta-hydroxydeacetyl baccatin III), 10-deacetyl baccatin III, baccatin V, taxol B (cephalomannine), taxol C, taxol D, taxol E, taxol F, taxol G, 7-xylosyl-10-deacetyl cephalomannine, 7-xylosyl-10-deacetyl paclitaxel, 10-deacetyl cephalomannine, 7-xylosyl-10-deacetyl taxol C, 10-deacetyl paclitaxel, 7-xylosyl paclitaxel, 10-deacetyl taxol C, 10-deacetyl-7-epi cephalomannine, 7-xylosyl taxol C, 10-deacetyl-7-epi paclitaxel, 7-epi cephalomannine, 7-epi paclitaxel, 7-O— methylthiomethyl paclitaxel, 7-deoxy docetaxel, and taxanime M. Taxanes may also comprise taxane mimics, which are compounds that may not be structurally similar, but which have a similar mechanism of action (i.e., bind beta-tubulin and block microtubule depolymerization). Taxane mimics include cyclostreptin, dictyostatin, discodermolide, eleutherobin, epothilones A/B, laulimalide, and peloruside.

(b) Cancers

Taxanes prevent cell proliferation by blocking cell mitosis and, thus, are anti-cancer or anti-neoplastic agents. Paclitaxel and docetaxel are widely used in the treatment of a variety of cancers. The cancers may be primary or metastatic; the cancers may be early stage or late stage. Taxanes may be used to treat breast cancer, ovarian cancer, non-small cell lung cancer (NSCLC, which includes squamous cell carcinoma, adenocarcinoma, and large cell carcinoma), small cell lung cancer, head and neck cancer, and Kaposi's sarcoma. Other cancers that may be treated with a taxane include, but are not limited to, bladder cancer, bone cancer, brain cancer, cervical cancer, colon cancer, duodenal cancer, endometrial cancer, esophageal cancer, eye cancer, gallbladder cancer, liver cancer, larynx cancer, lymphomas, melanoma, mouth cancer, pancreatic cancer, penal cancer, prostate cancer, rectal cancer, renal cancer, skin cancer, testicular cancer, thyroid cancer, and vaginal cancer. Taxanes may also be used to treat non-malignant neoplastic disorders, such as benign tumors of the breast, cervix, esophagus, lung, prostate, uterus, etc.

In one embodiment, the method of the invention may be used to determine the responsiveness of a breast cancer cell to a taxane. In another embodiment, the method of the invention may be used to determine the responsiveness of an ovarian cancer cell to a taxane. In a preferred embodiment, the method of the invention may be used to determine the responsiveness of a lung cancer cell to a taxane. In an exemplary embodiment, the method of the invention may be used to determine the responsiveness of a non-small cell lung cancer cell (NSCLC) to a taxane. The NSCLC may be a squamous cell carcinoma, an adenocarcinoma, or a large cell carcinoma.

(c) Molecular Markers

A plurality of molecular markers was identified by screening a human lung cancer cell line with a library of short hairpin RNA (shRNA) constructs targeting human tumor suppressor genes. shRNA produces highly stable, long-term gene silencing using an RNA interference (RNAi) mechanism. Cell survival in the presence and absence of a taxane revealed that some gene knockdowns decreased cell survival and other gene knockdowns increased cell survival in the presence of the drug (see Example 2). Changes in the expression of these marker genes in a cancer cell may be used to assess whether a cancer cell will respond therapeutically to a taxane. The panel of markers (see Table 1) comprises BRCA2, CDKN1C, CYLD, DCC, DMBT1, LZTS2, MSH2, PHB, SMAD4, ST14, ST18, TGFBR2, and VHL, which are tumor suppressor genes whose decreased expression in a cancer cell indicates that the cancer cell will respond therapeutically to a taxane. The panel of molecular markers also comprises CDKN2A, FOS, GLTSCR2, HIC1, LATS1, LATS2, LZTS1, PTEN, ST18, TP53, TP73, TUSC2, TUSC5, and WT1, which are tumor suppressor genes whose decreased expression in a cancer cell indicates that the cancer cell will not respond therapeutically to a taxane.

In one embodiment, the altered expression of one of the molecular markers of the invention may be used to determine whether a cancer cell will respond to a taxane. In other embodiments, the altered expression of 2, 3, 4, 5, 6, 7, 8, 9, or 10 of the molecular markers may be used to determine whether a cancer cell will respond to a taxane. In yet other embodiments, the altered expression of 11, 12, 13, 14, 15, 16, 17, 18, or 19 of the molecular markers may be used to determine whether a cancer cell will respond to a taxane. In still further embodiments, the altered expression of 20, 21, 22, 23, 24, 25, 26, or 27 of the molecular markers may be used to determine whether a cancer cell will respond to a taxane. One skilled in the art will appreciate that, generally, the more markers examined, the more accurate the determination of whether or not the cell will respond therapeutically to a taxane.

TABLE 1
Molecular Markers

| | | GenBank |
|---|---|---|
| Official | | Accession |
| Name | Gene Name | Number |
| BRCA2 | breast cancer 2, early onset | NM_000059 |
| CDKN1C | cyclin-dependent kinase inhibitor 1C | NM_000076 |
| CDKN2A | cyclin-dependent kinase inhibitor 2A | NM_058197 |
| CYLD | cylindromatosis (turban tumor syndrome) | NM_015247 |
| DCC | deleted in colorectal carcinoma | NM_005215 |
| DMBT1 | deleted in malignant brain tumors 1 | NM_004406 |
| FOS | FBJ murine osteosarcoma viral oncogene | NM_005252 |
| | homolog | |
| GLTSCR2 | glioma tumor suppressor candidate | NM_015710 |
| | region gene 2 | |
| HIC1 | hypermethylated in cancer 1 | NM_006497 |
| LATS1 | large tumor suppressor, homolog 1 | NM_004690 |
| LATS2 | large tumor suppressor, homolog 2 | NM_014572 |
| LZTS1 | leucine zipper, putative tumor | NM_021020 |
| | suppressor 1 | |
| LZTS2 | leucine zipper, putative tumor | NM_032429 |
| | suppressor 2 | |
| MSH2 | mutS homolog 2 | NM_000251 |
| NF1 | neurofibromin 1 (neurofibromatosis 1) | NM_000267 |
| PHB | prohibitin | NM_002634 |
| PTEN | phosphatase and tensin homolog | NM_000314 |
| SMAD4 | SMAD, mothers against DPP homolog 4 | NM_005359 |
| ST14 | suppression of tumorigenicity 14 | NM_021978 |
| ST18 | suppression of tumorigenicity 18 | NM_014682 |
| TGFBR2 | transforming growth factor, beta | NM_003242 |
| | receptor II | |
| TP53 | tumor protein p53 | NM_000546 |
| TP73 | tumor protein p73 | NM_005427 |
| TUSC2 | tumor suppressor candidate 2 | NM_007275 |
| TUSC5 | tumor suppressor candidate 5 | NM_172367 |
| VHL | von Hippel-Lindau tumor suppressor | NM_000551 |
| WT1 | Wilms tumor 1 | NM_024424 |

(d) Measuring Expression

Measuring the expression of the molecular marker or markers may be accomplished by a variety of techniques that are well known in the art. Expression may be monitored directly by detecting products of the molecular marker genes (i.e., mRNA or protein), or it may be assessed indirectly by detecting alterations in the DNA (e.g., amplification, methylation, etc.) that affect expression of the molecular marker genes. RNA, protein, or DNA may be isolated from cells of interest using techniques well known in the art and disclosed in standard molecular biology reference books, such as Ausubel et al., (2003) Current Protocols in Molecular Biology, John Wiley & Sons, New York, N.Y.

(i) Detecting RNA

Detection of the RNA products of the molecular marker genes may be accomplished by a variety of methods. Some methods are quantitative and allow estimation of the original levels of RNA between the cancer and control cells, whereas other methods are merely qualitative. Additional information regarding the methods presented below may be found in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. A person skilled in the art will know which parameters may be manipulated to optimize detection of the mRNA of interest.

Quantitative real-time PCR (QRT-PCR) may be used to measure the differential expression of a molecular marker in a cancer cell and a control cell. In QRT-PCR, the RNA template is generally reverse transcribed into cDNA, which is then amplified via a PCR reaction. The PCR amplification process is catalyzed by a thermostable DNA polymerase. Non-limiting examples of suitable thermostable DNA polymerases include Taq DNA polymerase, Pfu DNA polymerase, Tli (also known as Vent) DNA polymerase, Tfl DNA polymerase, and Tth DNA polymerase. The PCR process may comprise 3 steps (i.e., denaturation, annealing, and extension) or 2 steps (i.e., denaturation and annealing/extension). The temperature of the annealing or annealing/extension step can and will vary, depending upon the amplification primers. That is, their nucleotide sequences, melting temperatures, and/or concentrations. The temperature of the annealing or annealing/extending step may range from about 50° C. to about 75° C. The amount of PCR product is followed cycle-by-cycle in real time, which allows for determination of the initial concentrations of mRNA. The reaction may be performed in the presence of a dye that binds to double-stranded DNA, such as SYBR Green. The reaction may also be performed with a fluorescent reporter probes, such as TAQMAN® probes (Applied Biosystems, Foster City, Calif.) that fluoresce when the quencher is removed during the PCR extension cycle. Fluorescence values are recorded during each cycle and represent the amount of product amplified to that point in the amplification reaction. The cycle when the fluorescent signal is first recorded as statistically significant is the threshold cycle (Ct). To minimize errors and reduce any sample-to-sample variation, QRT-PCR is typically performed using an internal standard. The ideal internal standard is expressed at a constant level among different tissues, and is unaffected by the experimental treatment. Suitable internal standards include, but are not limited to, mRNAs for the housekeeping genes glyceraldehyde-3-phosphate-dehydrogenase (GAPDH) and beta-actin.

Reverse-transcriptase PCR (RT-PCR) may also be used to measure the differential expression of a molecular marker. As above, the RNA template is reverse transcribed into cDNA, which is then amplified via a typical PCR reaction. After a set number of cycles the amplified DNA products are typically separated by gel electrophoresis. Comparison of the relative amount of PCR product amplified in the different cells will reveal whether the molecular marker is differentially expressed in the cancer cell.

Differential expression of a molecular marker may also be measured using a nucleic acid microarray. In this method, single-stranded nucleic acids (e.g., cDNAs, oligonucleotides, etc.) are plated, or arrayed, on a solid support. The solid support may be a material such as glass, silica-based, silicon-based, a synthetic polymer, a biological polymer, a copolymer, a metal, or a membrane. The form or shape of the solid support may vary, depending on the application. Suitable examples include, but are not limited to, slides, strips, plates, wells, microparticles, fibers (such as optical fibers), gels, and combinations thereof. The arrayed immobilized sequences are generally hybridized with specific DNA probes from the cells of interest. Fluorescently labeled cDNA probes may be generated through incorporation of fluorescently labeled deoxynucleotides by reverse transcription of RNA extracted from the cells of interest. The probes are hybridized to the immobilized nucleic acids on the microchip under highly stringent conditions. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified molecular marker is thus determined simultaneously. Microarray analysis may be performed by commercially available equipment, following manufacturer's protocols, such as by using the Affymetrix GenChip technology, or Incyte's microarray technology.

Differential expression of a molecular marker may also be measured using Northern blotting. For this, RNA samples are first separated by size via electrophoresis in an agarose gel under denaturing conditions. The RNA is then transferred to a membrane, crosslinked, and hybridized, under highly stringent conditions, to a labeled DNA probe. After washing to remove the non-specifically bound probe, the hybridized labeled species are detected using techniques well known in the art. The probe may be labeled with a radioactive element, a chemical that fluoresce when exposed to ultraviolet light, a tag that is detected with an antibody, or an enzyme that catalyses the formation of a colored or a fluorescent product. A comparison of the relative amounts of RNA detected in the different cells will reveal whether the expression of the molecular marker is changed in the cancer cell.

Nuclease protection assays may also be used to monitor the differential expression of a molecular marker in cancer and control cells. In nuclease protection assays, an antisense probe hybridizes in solution to an RNA sample. The antisense probe may be labeled with an isotope, a fluorophore, an enzyme, or another tag. Following hybridization, nucleases are added to degrade the single-stranded, unhybridized probe and RNA. An acrylamide gel is used to separate the remaining protected double-stranded fragments, which are then detected using techniques well known in the art. Again, qualitative differences in expression may be detected.

Differential expression of a molecular marker may also be measured using in situ hybridization. This type of hybridization uses a labeled antisense probe to localize a particular mRNA in cells of a tissue section. The hybridization and washing steps are generally performed under highly stringent conditions. The probe may be labeled with a fluorophore or a small tag (such as biotin or digoxigenin) that may be detected by another protein or antibody, such that the labeled hybrid may be visualized under a microscope. The transcripts of a molecular marker may be localized to the nucleus, the cytoplasm, or the plasma membrane of a cell.

(ii) Detecting Protein

Detection of the protein products of the molecular markers may be accomplished by several different techniques, many of which are antibody-based. Additional information regarding the methods discussed below may be found in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize detection of the protein of interest.

An enzyme-linked immunosorbent assay or ELISA may be used to detect and quantitate protein levels. This method comprises preparing the antigen (i.e., protein of interest), coating the wells of a microtiter plate with the antigen, incubating with an antibody that recognizes the antigen, washing away the unbound antibody, and detecting the antibody-antigen complex. The antibody is generally conjugated to an enzyme, such as horseradish peroxidase or alkaline phosphatase, which generate calorimetric, fluorescent, or chemiluminescent products. An ELISA may also use two antibodies, one of which is specific to the protein of interest and the other of which recognizes the first antibody and is coupled to an enzyme for detection. Further, instead of coating the well with the antigen, the antibody may be coated on the well. In this case, a second antibody conjugated to a detectable compound is added following the addition of the antigen of interest to the coated well.

Relative protein levels may also be measured by Western blotting. Western blotting generally comprises preparing protein samples, using gel electrophoresis to separate the denatured proteins by mass, and probing the blot with antibodies specific to the protein of interest. Detection is usually accomplished using two antibodies, the second of which is conjugated to an enzyme for detection or another reporter molecule. Methods used to detect differences in protein levels include calorimetric detection, chemiluminescent detection, fluorescent detection, and radioactive detection.

Measurement of protein levels may also be performed using a protein microarray or an antibody microarray. In these methods, the proteins or antibodies are covalently attached to the surface of the microarray or biochip. The protein of interest is detected by interaction with an antibody, and the antibody/antigen complexes are generally detected via fluorescent tags on the antibody.

Relative protein levels may also be assessed by immunohistochemistry, in which a protein is localized in cells of a tissue section by its interaction with a specific antibody. The antigen/antibody complex may be visualized by a variety of methods. One or two antibodies may be used, as described above for ELISA. The detection antibody may be tagged with a fluorophore, or it may be conjugated to an enzyme that catalyzes the production of a detectable product. The labeled complex is typically visualized under a microscope.

(iii) Detecting Alterations in DNA

Changes in the expression of a molecular marker may also be assessed by detecting alterations in the DNA encoding a molecular marker gene. The DNA may be amplified, which is a process whereby the number of copies of a region of DNA or a gene is increased. Usually, the amount of RNA product is also increased, in proportion to the number of additional copies of DNA. Amplification of DNA may be detected by PCR techniques, which are well known in the art. Amplification of DNA may also be detected by Southern blotting, in which genomic DNA is hybridized to labeled probes under highly stringent conditions, and the labeled hybrids may be detected as described above for Northern blotting. Amplification of DNA may also be detected by comparative genomic hybridization (CGH). CGH is a type of in situ hybridization in which the gain, loss, or amplification of DNA is detected at the level of the chromosome. The method is based on the hybridization of fluorescently labeled cancer cell DNA (e.g., labeled with fluorescein) and normal DNA (e.g., labeled with rhodamine) to normal human metaphase chromosome preparations. Using epifluorescence microscopy and quantitative image analysis, regional differences in the fluorescence ratio of cancer vs. control DNA is detected and used to identify abnormal regions in the cancer cell genomic DNA.

Changes in the methylation status of DNA may also indicate changes in expression of a molecular marker. The regulatory region of a gene may be methylated, which entails the addition of a methyl group to the 5-carbon of cytosine in a CpG dinucleotide. Genes that are transcriptionally silent tend to have methylated or hypermethylated regulatory regions. Thus, demethylation of a molecular marker gene may lead to increased expression in a cancer cell. Likewise, methylation of a molecular marker gene may lead to decreased expression in a cancer cell. Changes in the methylation status of a molecular marker gene in a cancer cell relative to a control cell may be assessed using methylation-sensitive restriction enzymes to digest DNA followed by Southern detection or PCR amplification. Changes in the methylation status of a molecular marker may also be detected using a bisulfite reaction based method. For this, sodium bisulfite is used to convert unmethylated cytosines to uracils, and then the methylated cytosines are detected by methylation specific PCR (MSP).

Single nucleotide polymorphisms (SNPs) in the regulatory region of a molecular marker gene may also affect its level of expression. For example, an altered nucleotide may affect the binding of a transcription factor such that transcription is up-regulated or down-regulated. The presence of a particular SNP may be detected by DNA sequencing. A SNP may also be detected by selective hybridization to an oligonucleotide probe (i.e., it hybridizes to a sequence containing a particular SNP, but not to sequences without the SNP). A particular SNP may also be detected using a PCR based technique or an oligonucleotide microarray based assay.

(e) Measuring Expression in Cells

Expression of the molecular marker or markers will generally be measured in a cancer cell relative to a control cell. The cell may be isolated from a subject so that expression of the marker may be examined in vitro. The type of biopsy used to isolated cells can and will vary, depending upon the location and nature of the cancer.

A sample of cells, tissue, or fluid may be removed by needle aspiration biopsy. For this, a fine needle attached to a syringe is inserted through the skin and into the organ or tissue of interest. The needle is typically guided to the region of interest using ultrasound or computed tomography (CT) imaging. Once the needle is inserted into the tissue, a vacuum is created with the syringe such that cells or fluid may be sucked through the needle and collected in the syringe. A sample of cells or tissue may also be removed by incisional or core biopsy. For this, a cone, a cylinder, or a tiny bit of tissue is removed from the region of interest. This type of biopsy is generally guided by CT imaging, ultrasound, or an endoscope. Lastly, the entire cancerous tumor may be removed by excisional biopsy or surgical resection.

RNA, protein, or DNA may be extracted from the biopsied cells or tissue to permit analysis of the expression of a molecular marker using methods described above in section (I)(d). The biopsied cells or tissue may also be embedded in plastic or paraffin, from which nucleic acids may be isolated. The expression of a molecular marker may also be performed in the biopsied cells or tissue in situ (e.g., in situ hybridization, immunohistochemistry).

Expression of a molecular marker may also be examined in vivo in a subject. A particular mRNA or protein may be labeled with fluorescent dye, a bioluminescent marker, a fluorescent semiconductor nanocrystal, or a short-lived radioisotope, and then the subject may be imaged or scanned using a variety of techniques, depending upon the type of label.

(f) Selecting a Treatment

Once the responsiveness of a cancer to a taxane has been determined, an effective treatment may be selected for treating a subject with cancer. If the cancer is determined to be responsive to a taxane, then a treatment comprising a taxane may be given to the subject. If, however, the cancer is determined to be non-responsive to a taxane, then another treatment may be selected for the subject. Thus, determining the responsiveness of a cancer before administering a treatment regime would spare subjects from potentially toxic treatments that would have no clinically positive outcome.

If a taxane is determined to be beneficial for the subject, then a taxane treatment regime is appropriate. The subject will generally be a mammal, and preferably, a human. The cancer to be treated may be a cancer listed in section (I)(b). In one embodiment, the cancer to be treated may be a breast cancer. In another embodiment, the cancer to be treated may be an ovarian cancer. In a preferred embodiment, the cancer to be treated may be a lung cancer. In an exemplary embodiment, the lung cancer may be a non-small cell lung cancer (NSCLC). The NSCLC may be a squamous cell carcinoma, an adenocarcinoma, or a large cell carcinoma.

The taxane may comprise paclitaxel, docetaxel, derivatives, analogs, or mimics thereof, as detailed above in section (I)(a). The route of administration can and will vary depending upon the location and nature of the cancer. The route of administration may be intradermal, transdermal, parenteral, intravenous, intramuscular, intranasal, subcutaneous, percutaneous, intratracheal, intraperitoneal, intratumoral, oral, perfusion, lavage, or direct injection. The treatment regimen can and will vary, depending on the type of cancer, its location, its stage of progression, and the health and age of the subject.

The treatment may further comprise administering at least one additional chemotherapeutic agent. Suitable chemotherapeutic agents include alkylating agents, such as cyclophosphamide, ifosfamide, and mitomycin C; anthracyclines, such as doxorubicin and epirubicin; topoisomerase inhibitors, such as camptothecins and etoposide; anti-metabolites, such as edatrexate, 5-fluorouracil, gemcitabine, and methotrexate; platinum-based agents, such as carboplatin and cisplatin, vinca alkaloids, such as vinblastine and vindesine; cytoskeletal disruptors, such as vinorelbine; a nitrosourea, such as fotemustine. The taxane may also be combined with a protein kinase inhibitor, such as bevacizumab, cetuximab, gefitinib, imatinib, or trastazumab.

The treatment may further comprise administering an inhibitor of BRCA2, an inhibitor of CDKN1C, an inhibitor of CYLD, an inhibitor of DCC, an inhibitor of DMBT1, an inhibitor of LZTS2, an inhibitor of MSH2, an inhibitor of PHB, an inhibitor of SMAD4, an inhibitor of ST14, an inhibitor of ST18, an inhibitor of TGFBR2, and an inhibitor of VHL, as described below in section (III).

The combination treatment comprising taxane and another agent can and will vary depending upon the agents and the cancer to be treated. The taxane and the other agent may be administered simultaneously. Alternatively, the taxane (T) and the other agent (A) may be administered sequentially or alternatively, e.g., TTAA, AATT, TATA, ATAT, TAAT, or ATTA, etc.

(II) Kits for Determining Whether a Cancer Cell Will Respond to a Taxane

Another aspect of the invention is the provision of kits for determining the responsiveness of a cancer cell to a taxane compound. The kits comprise a plurality of agents for measuring the expression of at least one molecular marker of the invention in a cancer cell, wherein changes in the expression of the molecular marker in the cancer cell relative to a control cell indicate that the cancer will respond therapeutically to a taxane. The molecular markers comprise BRCA2, CDKN1C, CDKN2A, CYLD, DCC, DMBT1, FOS, GLTSCR2, HIC1, LATS1, LATS2, LZTS1, LZTS2, MSH2, NF1, PHB, PTEN, SMAD4, ST14, ST18, TGFBR2, TP53, TP73, TUSC2, TUSC5, VHL, and WT1.

The agents to be used in the measurement of the expression of the molecular marker can and will vary, depending upon the type of technique to be used. In one embodiment, the kit may comprise oligonucleotide primers for QRT-PCR. The kit may further comprise fluorescent reporter probes. The kit may also further comprise a reverse transcriptase, a Taq polymerase, and appropriate buffers and salts. In another embodiment, the kit may comprise antibodies for ELISAs. The kit may further comprise a substrate for detection of enzyme-conjugated antibodies.

(III) Composition and Method for Treating a Cancer

A further aspect of the invention provides a method for treating a cancer in a subject. The method comprises administering to the subject a composition comprising a taxane and an inhibitor of BRCA2, an inhibitor of CDKN1C, an inhibitor of CYLD, an inhibitor of DCC, an inhibitor of DMBT1, an inhibitor of LZTS2, an inhibitor of MSH2, an inhibitor of PHB, an inhibitor of SMAD4, an inhibitor of ST14, an inhibitor of ST18, an inhibitor of TGFBR2, or an inhibitor of VHL. Decreased expression of BRCA2, CDKN1C, CYLD, DCC, DMBT1, LZTS2, MSH2, PHB, SMAD4, ST14, ST18, TGFBR2, or VHL makes a cell more responsive to a taxane, thus, the inhibition of BRCA2, CDKN1C, CYLD, DCC, DMBT1, LZTS2, MSH2, PHB, SMAD4, ST14, ST18, TGFBR2, or VHL in a cell may make the cell more responsive to a taxane.

Expression of these molecular markers may be inhibited via activation of the RNAi pathway, which leads to reduced or inhibited translation of mRNA or mRNA degradation. RNAi may be induced by small interfering RNAs (siRNAs), shRNAs, or double-stranded RNAs. Expression of these markers may also be inhibited using antisense oligonucleotides. The antisense oligonucleotide may comprise standard or nonstandard deoxyribonucleotides or ribonucleotides linked via phosphodiester, phosphorothioate, or methylphosphonate bonds. The nucleotides may be modified with acetyl groups, amino groups, carboxyl groups, carboxymethyl groups, hydroxyl groups, methyl groups, phosphoryl groups, or thiol groups. For example, the nucleotide may be modified with 2′-O-alkyl groups on the sugar moieties, or C5-propyne or C5-alkyl groups on pyrimidine rings. The antisense oligonucleotide may also comprise morpholinos, which are synthetic molecules in which bases are attached to morpholino rings that are linked through phosphorodiamidate groups. The antisense oligonucleotide may also comprise alternative structural types, such as are peptide nucleic acids (PNA) or locked nucleic acids (LNA). The antisense oligonucleotide may also be a chimeric molecule that contains a mixture of the above-mentioned elements.

The function of BRCA2, CDKN1C, CYLD, DCC, DMBT1, LZTS2, MSH2, PHB, SMAD4, ST14, ST18, TGFBR2, or VHL may be inhibited by antibodies or fragments thereof. The antibodies may be polyclonal or monoclonal. The function of BRCA2, CDKN1C, CYLD, DCC, DMBT1, LZTS2, MSH2, PHB, SMAD4, ST14, ST18, TGFBR2, or VHL may also be inhibited by other proteins or protein fragments. As an example, SMAD4 is inhibited by Ski and Sno proteins (Wotton and Massague, (2001), Curr. Top. Microbiol. Immunol. 254:145-164).

The subject to be treated will generally be a mammal, and preferably, a human. The cancer to be treated will generally be a cancer listed in section (I)(b). In one embodiment, the cancer to be treated may be a breast cancer. In another embodiment, the cancer to be treated may be an ovarian cancer. In a preferred embodiment, the cancer to be treated may be a lung cancer. In an exemplary embodiment, the lung cancer may be a non-small cell lung cancer (NSCLC). The NSCLC may be a squamous cell carcinoma, an adenocarcinoma, or a large cell carcinoma. The taxane may comprise paclitaxel, docetaxel, derivatives, analogs, or mimics thereof, as detailed in section (I)(a). The treatment may further comprise administering at least one additional chemotherapeutic agent. Suitable chemotherapeutic agents and treatment regimes are presented above in section (I)(f).

### DEFINITIONS

The term “cancer,” as used herein, refers to the physiological condition in mammals that is typically characterized by unregulated cell proliferation, and the ability of those cells to invade other tissues. Cancer is synonymous with malignant neoplasm.

A “carcinoma” is a cancer that arises from epithelial cells.

The term “expression,” as used herein, refers to the conversion of the DNA sequence information into messenger RNA (mRNA) or protein. Expression may be monitored by measuring the levels of full-length mRNA, mRNA fragments, full-length protein, or protein fragments. Expression may also be inferred by assessing alterations in the DNA relative to a control state. Alterations in DNA that affect expression include amplification (increased copy number) of the DNA, changes in the methlyation status of the regulatory region of a gene, or single nucleotide polymorphisms in the regulatory region of a gene.

The term “hybridization,” as used herein, refers to the process of annealing or base-pairing via specific hydrogen bonds between two complementary single-stranded nucleic acids. The “stringency of hybridization” is determined by the conditions of temperature and ionic strength. Nucleic acid hybrid stability is expressed as the melting temperature or Tₘ, which is the temperature at which the hybrid is 50% denatured under defined conditions. Equations have been derived to estimate the Tₘ of a given hybrid; the equations take into account the G+C content of the nucleic acid, the length of the hybridization probe, etc. (e.g., Sambrook et al, 1989, chapter 9). To maximize the rate of annealing of the probe with its target, hybridizations are generally carried out in solutions of high ionic strength (6×SSC or 6×SSPE) at a temperature that is about 20-25° C. below the Tₘ. If the sequences to be hybridized are not identical, then the hybridization temperature is reduced 1-1.5° C. for every 1% of mismatch. In general, the washing conditions are as stringent as possible (i.e., low ionic strength at a temperature about 12-20° C. below the calculated Tₘ). As an example, highly stringent conditions typically involve hybridizing at 68° C. in 6×SSC/5×Denhardt's solution/1.0% SDS and washing in 0.2×SSC/0.1% SDS at 65° C. The optimal hybridization conditions generally differ between hybridizations performed in solution and hybridizations using immobilized nucleic acids. One skilled in the art will appreciate which parameters to manipulate to optimize hybridization.

The term “neoplasm,” as use herein, refers to an abnormal, disorganized growth in a tissue or organ, usually forming a distinct mass. Such a growth is called a neoplasm, also known as a “tumor.” The neoplasm or tumor may be benign or malignant. A malignant neoplasm (or cancer) is characterized by uncontrolled cell proliferation and the ability of those cells to invade other tissues.

The term “nucleic acid,” as used herein, refers to sequences of linked nucleotides. The nucleotides may be deoxyribonucleotides or ribonucleotides, they may be standard or non-standard nucleotides; they may be modified or derivatized nucleotides; they may be synthetic analogs. The nucleotides may be linked by phosphodiester bonds or non-hydrolyzable bonds. The nucleic acid may comprise a few nucleotides (i.e., oligonucleotide), or it may comprise many nucleotides (i.e., polynucleotide). The nucleic acid may be single-stranded or double-stranded.

As used herein, a “therapeutic response” or a “response” to a taxane means that the taxane affects a cell by blocking cell division, such that the rate of cell growth slows or stops and/or the cell dies.

The phrases “treatment for cancer,” “treating a cancer,” or “cancer treatment,” as used herein, refer to the administration of a therapeutic agent that kills the cancer cells, induces apoptosis in the cancer cells, reduces the growth rate of the cancer cells, reduces the incidence or number of metastases, reduces the tumor size, inhibits the tumor growth, prevents or inhibits the progression of the cancer, or increases the lifespan of a subject with cancer.

As various changes could be made in the above compounds, methods, and products without departing from the scope of the invention, it is intended that all matter contained in the above description and in the examples given below, shall be interpreted as illustrative and not in a limiting sense.

### EXAMPLES

The following examples illustrate the identification of the molecular markers whose alterations in expression reveal whether a lung cancer cell will respond to a taxane.

### Example 1

### MDR1 Knockdown Cells have Increased Sensitivity to Paclitaxel

Introduction. Prior to screening for genes that confer altered sensitivity to paclitaxel, the MDR1 gene was silenced in a human lung cancer cell line. Since MDR1 encodes P-glycoprotein, a human multi-drug resistant transporter, the MDR1 knockdown cells should be more responsive to paclitaxel. MDR1 was silenced using short hairpin RNA (shRNA) in lentiviral vectors.

Cell Culture. The human lung adenocarcinoma cell line A549 was obtained from ATCC (Manassas, Va.). The cell line was cultured in F12 Ham's media supplemented with 10% v/v fetal bovine serum, 4 mM final L-glutamine, penicillin and streptomycin (all from Sigma-Aldrich, St. Louis) in T75 cm₂ cell culture flasks, at 37° C. and 5% CO₂. At ˜80% confluency, the cells are trypsinized and reseeded into 96-well plates for assay.

Plasmid Midiprep. Isolation of the plasmid DNA from MDR1 constructs was performed using GenElute HP Plasmid Midiprep kit (Sigma-Aldrich, St. Louis, Mo.), following the appropriate protocol in the instruction manual. DNA was normalized to 20 μl/mL in a 1:10 dilution of DNA and TE, and analyzed using SoftMaxPro computer software.

Transfection. FuGENE6 Transfection Reagent was used, along with packaging construct (pDelta 8.9) and envelope construct (pCMV-VSV-G) in serum free DME media for all transfections. Virus particles were harvested at 40 and 48 hours post-transfection, yielding approximately 200 μl per sample. A p24 assay was performed to test for quality, using an HIV-1 p24 Antigen ELISA manual kit (Gentaur, Brussels, Belgium).

Infection. A549 cells were infected at a MOI (multiplicity of infection) of 10. Approximately 40,000 cells/well were seeded in a 24-well plate. Empty vector (pLKO.1) was a positive control for puromycin selection as well as negative control for MDR1 knockdown. Four replicates of each construct were infected and duplicates were made of the control and blank wells. The final concentration of polybrene used was 8 μg/mL, and the cells were selected with 3 μg/mL of puromycin at 48 hours post-infection.

Quantitative Real-Time PCT (QRT-PCR). RNA from the infected A549 cell lines was harvested using GenElute Mammalian Total RNA Kit (Sigma-Aldrich, St. Louis, Mo.). TaqMan Gene Expression Assays primer and probe, ABCB1, were used (Applied Biosystems, Foster City, Calif.). MDR1 primers were 5′-labeled with FAM reporter dye and 3′-labeled with fluorescent quencher. QRT-PCR was preformed using a Master Mix kit prepared with Sigma's quantitative RT-PCR ReadyMix (QRO200) supplemented with MgCl₂ and water. Reference dye was also included as an internal control for fluorescence. 20 μl total reactions were set up, using 4 μl of RNA. All reactions were run and analyzed with the Mx3000 qPCR system and software (Stratagene, La Jolla, Calif.). Reaction conditions were set up at: 15 min at 42° C., 3 min at 94° C., and 45 cycles of 15 s at 94° C. and 1 min at 60° C. mRNA expression from the MDR1 constructs were compared against GAPDH mRNA for quantification. Assays were preformed in triplicate (including the empty vector construct), as well as two no-template controls, and one with no reverse transcriptase mix. Values are expressed with pLKO.1 expression normalized to 100%.

Paclitaxel Exposure and Cytotoxicity Assay. Cells were plated overnight in 96-well plates at 40,000 cells/cm². Then increasing concentrations of paclitaxel in F12 Ham's media replaced the normal media, and the cells were cultured for 24 hours. In vitro cytotoxicity was performed using a Quick Cell Proliferation Assay Kit (BioVision, Mountain View, Calif.). For this, 10 μl of WST-1 was added to each well and the plates were incubated an additional 4 hours. The formazan dye produced by live cells was read by a spectrophotometer for absorbance at 450 nm using SoftMax Pro software. Absorbance measurements were normalized by subtracting the value of blank wells from the treated wells.

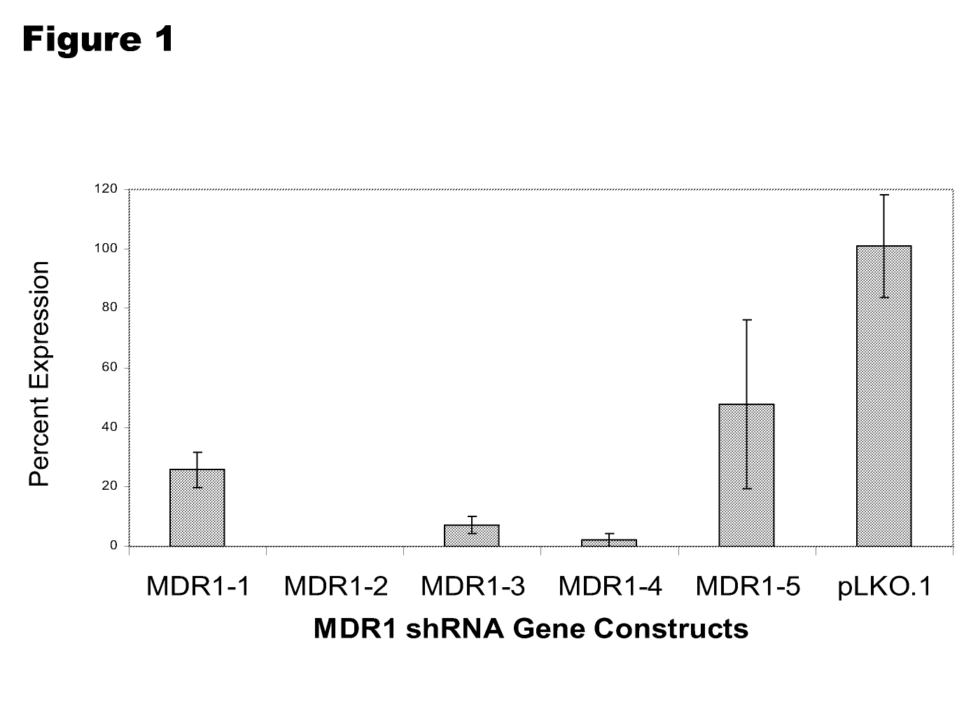

Results. MDR1 shRNA lentiviruses were produced and utilized to infect A549 cells. Each MDR1 construct: MDR1-1 (TRCN0000059683), MDR1-2 (TRCN0000059684), MDR1-3 (TRCN0000059685), MDR1-4 (TRCN0000059686), and MDR1-5 (TRCN0000059687) was measured by means of QRT-PCR and shown to be effective in knocking down transcription of the MDR1 gene (FIG. 1). Threshold values for the MDR1-2 construct were below the level of detection. All of the values were normalized to control cells, which were transduced with the empty vector, pLKO.1.

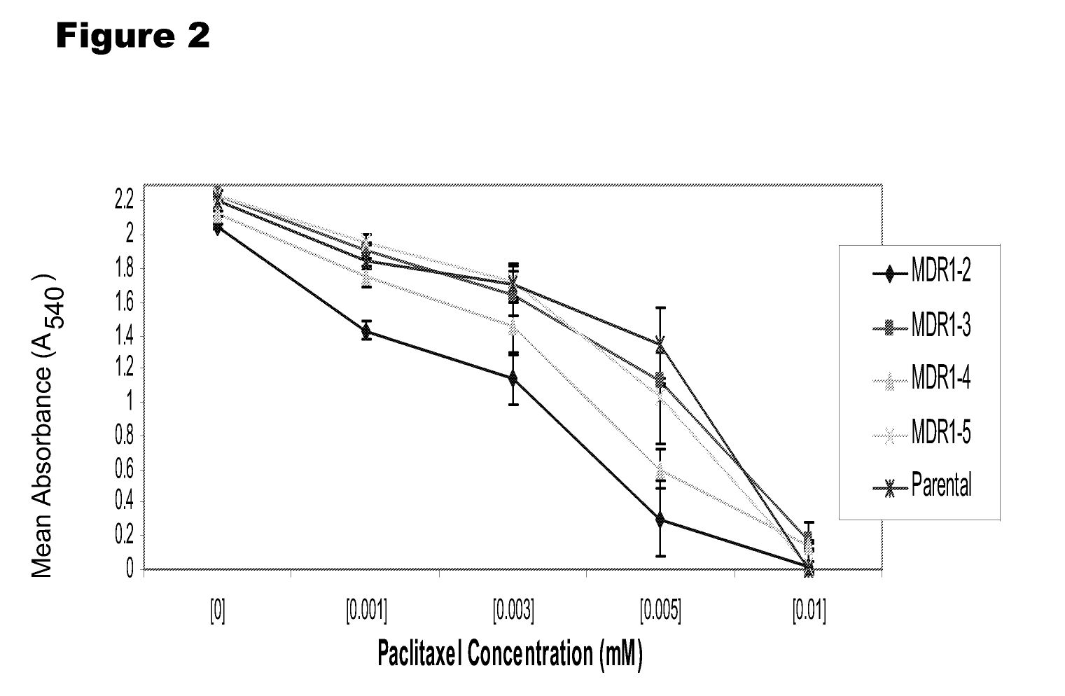

The cells were exposed to increasing concentration of pactitaxel (0, 1 μM, 3 μM, 5 μM, 7 μM, and 10 μM) for 24 hours, after which cell death assays were performed. The MDR1 knockdown cells were more sensitive to paclitaxel than the control cells (FIG. 2). MDR1-2, the construct demonstrating the greatest knockdown, was then used as a positive control for sensitizing cells to paclitaxel.

### Example 2

### Reverse Infection with a shRNA Tumor Suppressor Panel Identifies Markers to Assess Paclitaxel Sensitivity

Introduction. A459 cells were infected with lentiviral vectors containing shRNAs targeted to tumor suppressor genes. The infected cells were then grown in the presence or absence of paclitaxel, and the ratio of cellular proliferation was calculated. This analysis lead to the identification of genes, that when down-regulated, altered the responsiveness of a lung cancer cell to paclitaxel.

Reverse Infection. Reverse infection was performed on A549 cells with 5 μl of virus from a tumor suppressor panel (MISSION™ TRC-Hs 1.0 shRNA Human Tumor Suppressor Gene Family, Sigma-Aldrich, St. Louis) at a seeding density of 16,000 cells/well in a 96-well plate. Triplicates of pLKO.1 virus was included as a negative control, and MDR1-2 virus served as a positive control. The final concentration of polybrene was 8 μg/mL, and 3 μg/mL of puromycin was used to select cells at 48 hours post-infection.

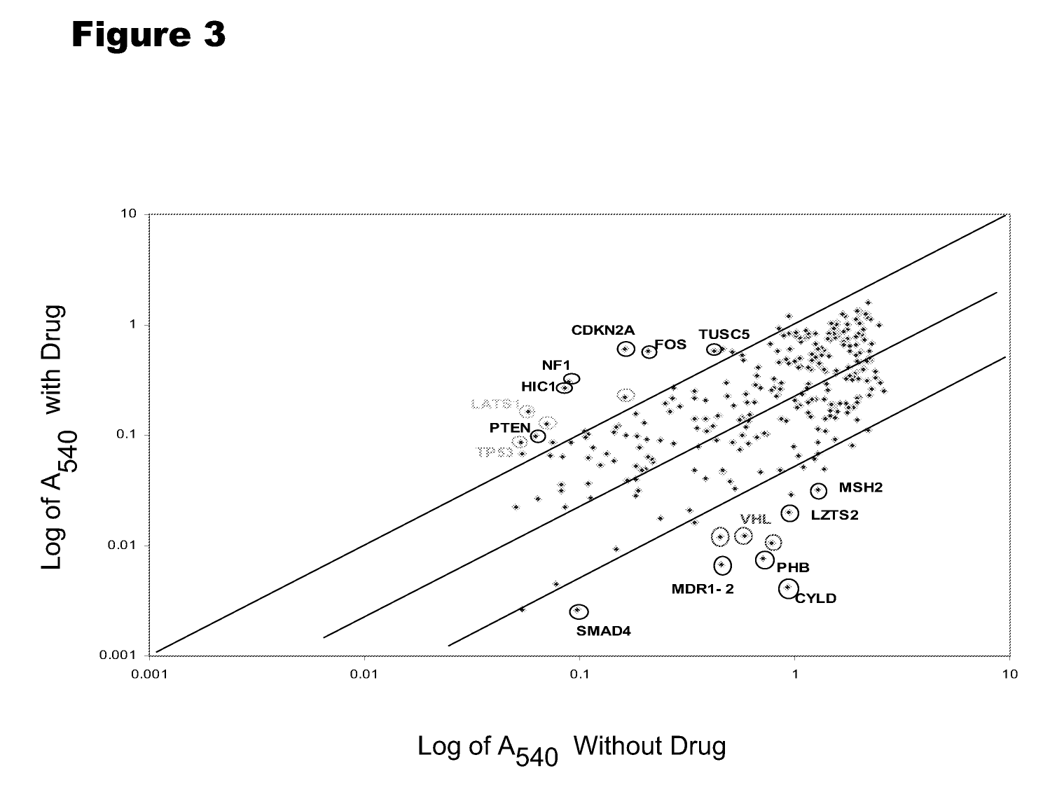

Results. A549 cells were transduced with 337 viruses targeting 74 tumor suppressor genes. The infected cells were then grown in the absence or presence of 5 μM of paclitaxel for 24 hours, and then cytotoxicity assays were performed (as described in Example 1). A plot of cell survival in the absence or presence of 5 μM of paclitaxel for 24 hours is shown in FIG. 3. The down-regulation of some genes increased cell survival (points above the lines) and the down-regulation of other genes decreased cell survival (points below the lines) under these conditions.

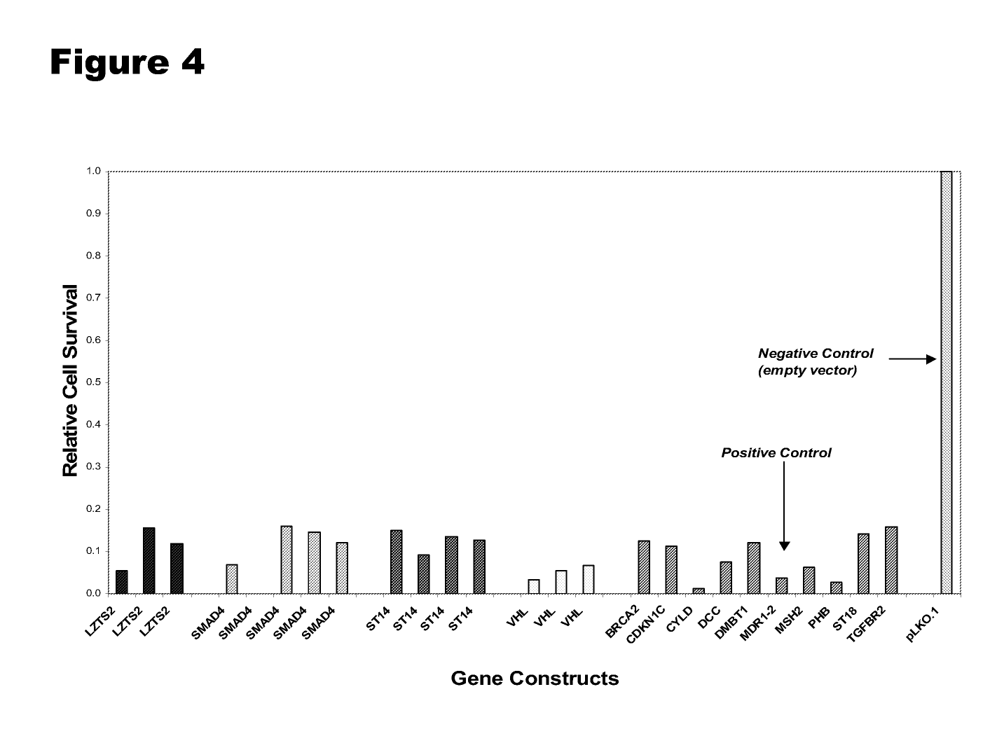

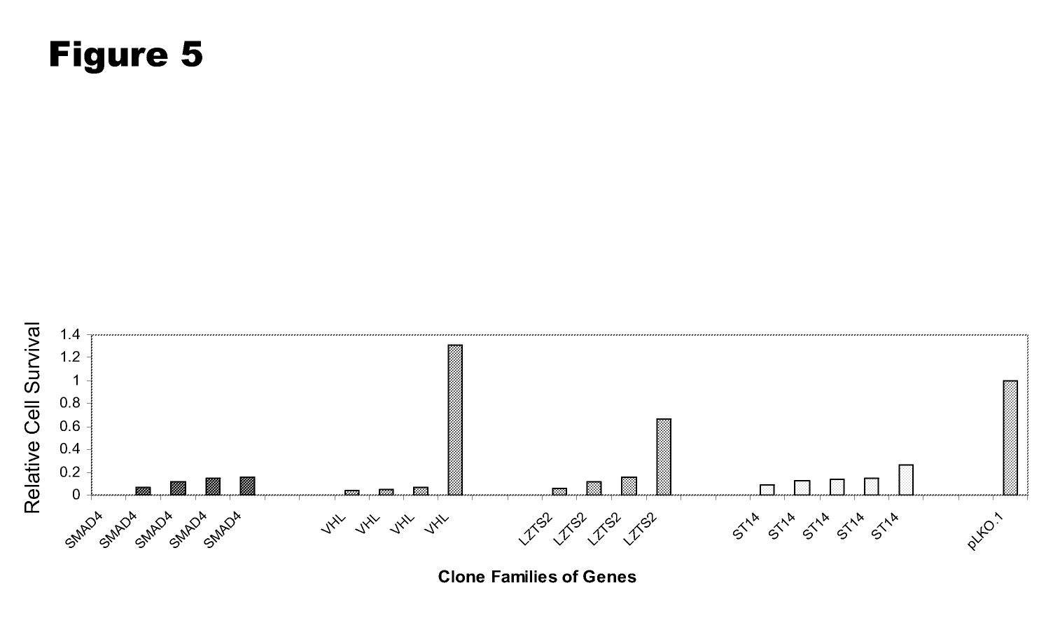

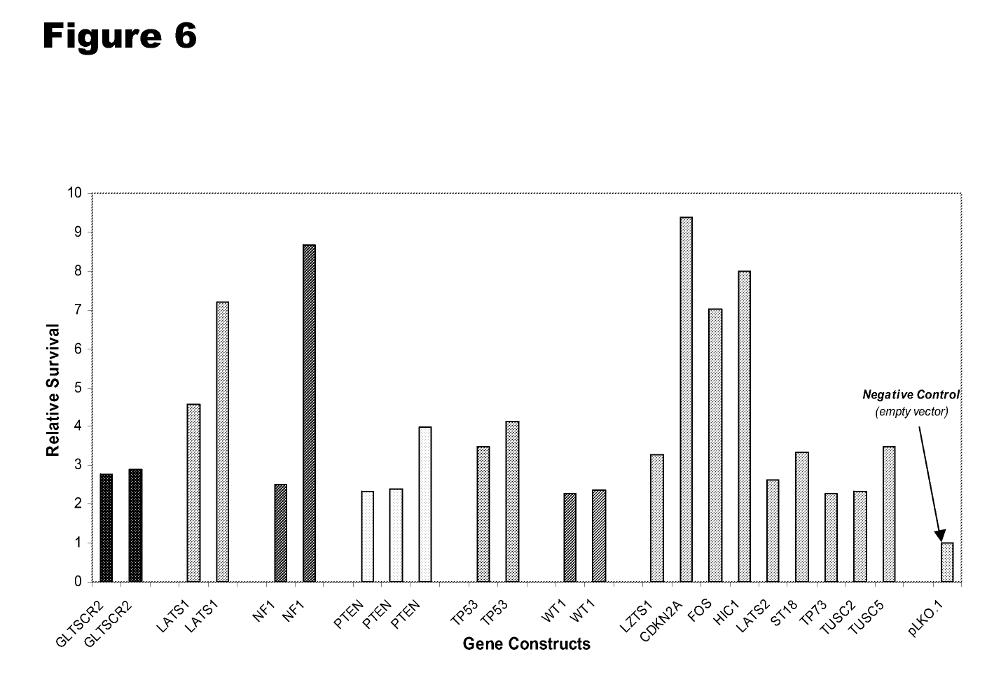

The genes whose down-regulation altered the cells' responsiveness to paclitaxel were identified (see Table 2). The ratios of cellular proliferation were calculated for cells containing these down-regulated genes, and are presented in FIGS. 4-6. Knockdown of BRCA2, CDKN1C, CYLD, DCC, DMBT1, LZTS2, MSH2, PHB, SMAD4, ST14, ST18, TGFBR2, or VHL increased the cells' responsiveness to paclitaxel, while knockdown of CDKN2A, FOS, GLTSCR2, HIC1, LATS1, LATS2, LZTS1, PTEN, ST18, TP53, TP73, TUSC2, TUSC5, or WT1 provided a protective effect. In many cases, there were multiple constructs to a single gene that yielded positive hits (see Table 2). The multiple constructs to a single gene generally affected cell survival in the same direction (see FIGS. 4-6).

TABLE 2
shRNA Constructs

| Target Gene | TRC number |
|---|---|
| BRCA2 | TRCN0000009825 |
| CDKN1C | TRCN0000039679 |
| CDKN2A | TRCN0000039748 |
| CYLD | TRCN0000039628 |
| DCC | TRCN0000039816 |
| DMBT1 | TRCN0000038730 |
| FOS | TRCN0000016007 |
| GLTSCR2 | TRCN0000038089 |
| | TRCN0000038090 |
| HIC1 | TRCN0000014634 |
| LATS1 | TRCN0000001777 |
| | TRCN0000001778 |
| LATS2 | TRCN0000000883 |
| LZTS1 | TRCN0000015649 |
| LZTS2 | TRCN0000021128 |
| | TRCN0000021126 |
| | TRCN0000021127 |
| MSH2 | TRCN0000039668 |
| NF1 | TRCN0000039715 |
| | TRCN0000039716 |
| PHB | TRCN0000029204 |
| PTEN | TRCN0000002746 |
| | TRCN0000002748 |
| | TRCN0000002747 |
| SMAD4 | TRCN0000040031 |
| | TRCN0000040030 |
| | TRCN0000040028 |
| | TRCN0000040029 |
| | TRCN0000040032 |
| ST14 | TRCN0000038050 |
| | TRCN0000038051 |
| | TRCN0000038052 |
| | TRCN0000038053 |
| ST18 | TRCN0000013498 |
| | TRCN0000013502 |
| TGFBR2 | TRCN0000040010 |
| TP53 | TRCN0000003753 |
| | TRCN0000003754 |
| TP73 | TRCN0000006508 |
| TUSC2 | TRCN0000038087 |
| TUSC5 | TRCN0000038123 |
| VHL | TRCN0000039626 |
| | TRCN0000039624 |
| | TRCN0000039625 |
| WT1 | TRCN0000040067 |
| | TRCN0000001117 |

Conclusions. A panel of tumor suppressor genes was identified that will predict the responsiveness of a lung tumor cell to paclitaxel. Decreased expression of BRCA2, CDKN1C, CYLD, DCC, DMBT1, LZTS2, MSH2, PHB, SMAD4, ST14, ST18, TGFBR2, or VHL indicate that a lung cancer cell will be responsive to paclitaxel. And decreased expression of CDKN2A, FOS, GLTSCR2, HIC1, LATS1, LATS2, LZTS1, PTEN, ST18, TP53, TP73, TUSC2, TUSC5, or WT1 indicate that a lung cancer cell will not be responsive to paclitaxel.

What is claimed is:

1. A method for determining whether a lung cancer will respond to a taxane, the method comprising:
a. measuring the expression of at least one molecular marker in a cell from the cancer, wherein the molecular marker is selected from the group consisting of BRCA2, CDKN1C, CDKN2A, CYLD, DCC, DMBT1, FOS, GLTSCR2, H1C1, LATS1, LATS2, LZTS1, LZTS2, MSH2, NF1, PHB, PTEN, SMAD4, ST14, ST18, TGFBR2, TP53, TP73, TUSC2, TUSC5, VHL, and WT1; and
b. assessing the responsiveness of the cancer to the taxane based upon expression of the molecular marker in the cancer cell relative to a control cell.

2. The method of claim 1, wherein the decreased expression of BRCA2, CDKN1C, CYLD, DCC, DMBT1, LZTS2, MSH2, PHB, SMAD4, ST14, ST18, TGFBR2, or VHL in the cancer cell indicates that the cancer will respond to a taxane, and the decreased expression of CDKN2A, FOS, GLTSCR2, HIC1, LATS1, LATS2, LZTS1, PTEN, ST18, TP53, TP73, TUSC2, TUSC5, or WT1 in the cancer cell indicates that the cancer will not respond to a taxane.

3. The method of claim 1, wherein measuring the expression of the molecule marker comprises detecting messenger RNA by a method selected from the group consisting of quantitative real time RCR (QRT-PCR), reverse transcriptase PCR(RT-PCR), nucleic acid microarray, in situ hybridization, and Northern blotting.

4. The method of claim 1, wherein measuring the expression of the molecule marker comprises detecting protein by a method selected from the group consisting of enzyme-linked immunosorbent assay, Western blotting, immunohistochemistry, protein microarray, and antibody microarray.

5. The method of claim 1, wherein measuring the expression of the molecular marker comprises detecting alterations in DNA due to a process selected from the group consisting of DNA amplification, DNA methylation/demethylation, and single nucleotide polymorphisms.

6. The method of claim 1, wherein the lung cancer is a non-small cell lung cancer selected from the group consisting of squamous cell carcinoma, adenocarcinoma, and large cell carcinoma.

7. The method of claim 1, wherein the expression of the molecular marker is measured in the cell in vitro or in vivo.

8. The method of claim 7, wherein the in vitro cell is in a sample obtained by a method selected from the group consisting of a needle aspiration biopsy, an incisional biopsy, and an excisional biopsy.

9. The method of claim 1, wherein the taxane is selected from the group consisting of paclitaxel, docetaxel, derivatives, and analogs thereof.

10. The method of claim 1, wherein the expression of the molecular marker is measured by QRT-PCR in a biopsied cell from a non-small cell lung cancer.

11. The method of claim 1, wherein the method is used to select an effective treatment for a subject with lung cancer, the method further comprising administering the taxane to the subject if the cancer is determined to be responsive, or administering an alternate treatment to the subject if the cancer is determined to be non-responsive.

12. The method of claim 11, wherein the subject is a human.

13. The method of claim 11, wherein the lung cancer is a non-small cell lung cancer selected from the group consisting of squamous cell carcinoma, adenocarcinoma, and large cell carcinoma.

14. The method of claim 11, wherein the taxane is selected from the group consisting of paclitaxel, docetaxel, derivatives, and analogs thereof.

15. The method of claim 14, wherein the method further comprises administering a chemotherapeutic agent selected from the group consisting of carboplatin, cisplatin, etoposide, ifosfamide, mitomycin, vinblastine, and vindesine.

16. A kit for determining whether a lung cancer will respond to a taxane, the kit comprising a plurality of agents for measuring the expression of at least one molecular marker in a cell from the lung cancer, wherein the expression of the molecular marker is altered in the cancer cell relative to a control cell.

17. The kit of claim 16, wherein the molecular marker is selected from the group consisting of BRCA2, CDKN1C, CDKN2A, CYLD, DCC, DMBT1, FOS, GLTSCR2, H1C1, LATS1, LATS2, LZTS1, LZTS2, MSH2, NF1, PHB, PTEN, SMAD4, ST14, ST18, TGFBR2, TP53, TP73, TUSC2, TUSC5, VHL, and WT1.

18. The kit of claim 16, wherein the plurality of agents comprise oligonucleotide primers for QRT-PCR.

19. The kit of claim 18, further comprising at least one fluorescent reporter probe.

20. The kit of claim 16, wherein the plurality of agents comprise antibodies for enzyme-linked immunosorbent assays.

21. A plurality of molecular markers for determining a treatment for lung cancer, the molecular markers comprising BRCA2, CDKN1C, CDKN2A, CYLD, DCC, DMBT1, FOS, GLTSCR2, H1C1, LATS1, LATS2, LZTS1, LZTS2, MSH2, NF1, PHB, PTEN, SMAD4, ST14, ST18, TGFBR2, TP53, TP73, TUSC2, TUSC5, VHL, and WT1.
